(19) United States
(12) Patent Application Publication
(10) Pub. No.: US 2025/0302354 A1
(43) Pub. Date: Oct. 2, 2025

(54) COGNITIVE FUNCTION ESTIMATION DEVICE, COGNITIVE FUNCTION ESTIMATION METHOD, AND RECORDING MEDIUM

(71) Applicant: NEC Corporation, Tokyo (JP)

(72) Inventors: Masashi USAMI, Tokyo (JP); Terumi UMEMATSU, Tokyo (JP)

(73) Assignee: NEC Corporation, Tokyo (JP)

(21) Appl. No.: 19/076,059

(22) Filed: Mar. 11, 2025

(30) Foreign Application Priority Data
Mar. 26, 2024 (JP) 2024-049287

(51) Int. Cl. A61B 5/16; G06T 7/00; G06V 10/75; G06V 10/764; G06V 20/40; G06V 40/16

(52) U.S. Cl. CPC A61B 5/165; G06T 7/0012; G06V 10/751; G06V 10/764; G06V 20/41; G06V 20/46; G06V 40/171; G06V 40/176; G06T 2207/10016; G06T 2207/20081; G06T 2207/30201

(57) ABSTRACT

A cognitive function estimation device cognitive function estimation device acquires a video of a target person. The cognitive function estimation device detects a specific body part forming a body of the target person from the video, and acquire detection information concerning the body part. The cognitive function estimation device classifies each section of the video for each state by determining the state of the target person based on the detection information. The cognitive function estimation device calculates, for each state, a variation amount of a specific body part in each section of the video which is classified, and calculate features associated to the variation amount for each state. The cognitive function estimation device calculates comparison features, which are features related to comparison between states, by comparing features of respective states.

### TECHNICAL FIELD

This disclosure relates to a technique for supporting estimation of a cognitive function of a target person.

### BACKGROUND ART

The number of persons with dementia in Japan is increasing year by year, and it is said that the number of persons with dementia is also referred to as about 7 million in 2025. Early detection of dementia can to some extent reduce its progression and improve a state, but elderly people in single-person households, for example, are likely to be unaware that they themselves are developing dementia. Also, in general, a brain image test and a cognitive function test to detect dementia are expensive, time-consuming and not easy.

Despite the fact that the number of healthcare workers is unlikely to increase dramatically due to the declining birthrate and aging population, the number of dementia patients is highly likely to increase. Therefore, a system which detects dementia patients early and easily has become a social necessity.

Conventionally, a system for detecting a dementia using a facial image or a video and features extracted from the facial image or the video is known. Patent Document 1 describes a cognitive function estimation system in which an emotion level of a target person is estimated using a CNN (Convolutional Neural Network) model from image data, and a cognitive function is estimated from a change in the emotion level.
Patent Document 1: Japanese Patent Application Laid-Open under No. 2021-58231

### SUMMARY

Although an expression intensity and an expression frequency of emotion is said to be related to a cognitive function, a conventional cognitive function estimation system may not provide a good basis for judging the cognitive function due to individual differences and the individuality in the way the CNN model used to extract emotions produces scores. Also, an expression of emotion in a state of a natural conversation can swing widely depending on the compatibility of the conversation partner and the choice of topic. In other words, expressions of emotion are difficult to control, and results may change depending on a topic or a mood of that day, or an emotion desired to be analyzed may not have occurred during the conversation in the first place. Therefore, it is difficult to use the expression of emotion for the detection of the cognitive function because it may lack accuracy.

One object of the present disclosure is to support to estimate the cognitive function using a video of the target person.

According to an example aspect of the present invention, there is provided a cognitive function estimation device comprising:
at least one memory configured to store instructions; and
at least one processor configured to execute the instructions to:
acquire a video of a target person;
detect a specific body part forming a body of the target person from the video, and acquire detection information concerning the body part;
classify each section of the video for each state by determining the state of the target person based on the detection information;
calculate, for each state, a variation amount of a specific body part in each section of the video which is classified, and calculate features associated to the variation amount for each state; and
calculate comparison features, which are features related to comparison between states, by comparing features of respective states.

According to another example aspect of the present invention, there is provided a cognitive function estimation method performed by a cognitive function estimation device, comprising:
acquiring a video of a target person;
detecting a specific body part forming a body of the target person from the video, and acquire detection information concerning the body part;
classifying each section of the video for each state by determining the state of the target person based on the detection information;
calculating, for each state, a variation amount of a specific body part in each section of the video which is classified, and calculating features associated to the variation amount for each state; and
calculating comparison features, which are features related to comparison between states, by comparing features of respective states.

According to still another example aspect of the present invention, there is provided a program causing a computer to execute processing of:
acquiring a video of a target person;
detecting a specific body part forming a body of the target person from the video, and acquire detection information concerning the body part;
classifying each section of the video for each state by determining the state of the target person based on the detection information;
calculating, for each state, a variation amount of a specific body part in each section of the video which is classified, and calculating features associated to the variation amount for each state; and
calculating comparison features, which are features related to comparison between states, by comparing features of respective states.

### Effect

According to the present disclosure, it is possible to support to estimate the cognitive function using a video of the target person.

### BRIEF DESCRIPTION OF THE DRAWINGS

FIG. 1 illustrates an example of a schematical configuration a cognitive function estimation system.

FIG. 2 illustrates an example of a hardware configuration of a cognitive function estimation device.

FIG. 3 is a block diagram illustrating an example of a functional configuration of the cognitive function estimation device.

FIG. 4 is a diagram schematically representing a cognitive function estimation process.

FIG. 5 is a diagram illustrating feature points of components of a face with black dots.

FIG. 6 illustrates examples in the facial expressions of the target person in a standby state and a response state.

FIG. 7 is a flowchart of a recognition function estimation process.

FIG. 8 is a diagram schematically illustrating the recognition function estimation process using comparison features for a plurality of types for each state.

### EXAMPLE EMBODIMENTS

Preferred example embodiments of the present disclosure will be described with reference to the accompanying drawings.

### First Example Embodiment

### System Configuration

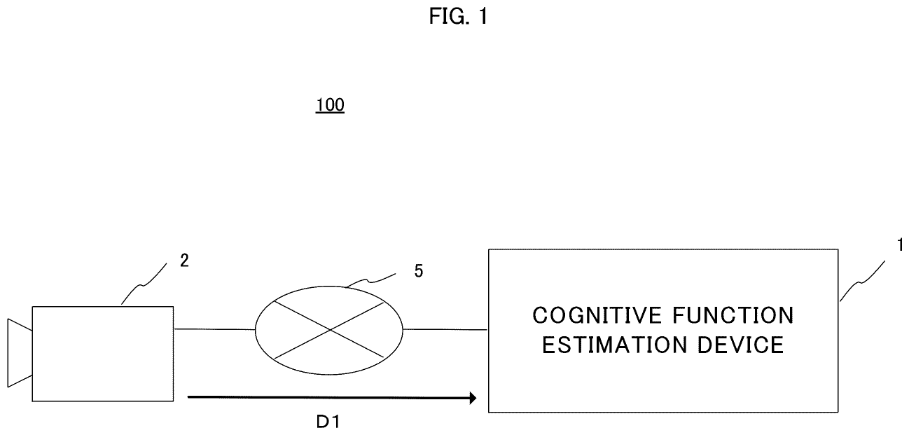

FIG. 1 is an example of a schematic configuration of a cognitive function estimation system 100 to which a cognitive function estimation device of the present disclosure is applied. The cognitive function estimation system 100 is a system that supports estimation of a cognitive function of a target person using video taken of the target person.

In the cognitive function estimation system 100, a cognitive function estimation device 1 and a camera 2 are communicably connected through a network 5 such as the Internet. The camera 2 captures conversations between a healthcare worker such as a doctor, and the target person whose cognitive function is to be estimated, and transmits still image data or video data captured as a video D1, to the cognitive function estimation device 1. The target person is, for instance, the elderly who are suspected of having a cognitive decline. Although it is desirable that the video D1 shows both the healthcare worker and the target person, the video D1 can be applied as long as the video D1 is an image of the target person during the conversation and shows a specific part of the body used for the cognitive function estimation. The cognitive function estimation device 1 is an information process device that processes, stores and transmits various data. The cognitive function estimation device estimates the cognitive function of the target person by analyzing the video D1.

In the present disclosure, the cognitive function estimation device 1 acquires the video D1 from the camera 2 through the network 5, but is not limited thereto. For instance, the video D1 may be acquired without using the network 5 through an external storage such as a USB (Universal Serial Bus) memory. A method by which the cognitive function estimation device 1 acquires the video D1 can be set arbitrarily. Furthermore, a person interacting with the target person is not limited to the healthcare worker, but may be a family member etc., for instance.

In the present disclosure, the cognitive function estimation system 100 outputs a classification based on a value corresponding to a score of mini-mental state examination (MMSE) which is one of evaluations of the cognitive function, or a value corresponding to the score of the MMSE, as an estimation result of the cognitive function. The MMSE is a widely used dementia test consisting of 11 items, including time orientation, place orientation, immediate and delayed recall of three words, calculation, object naming, sentence repetition, three-step verbal commands, written command following, sentence writing, and figure copying. It is a cognitive function test with a maximum score of 30 points. In addition, the classification based on the score of the MMSE score equivalent refers to a classification in which the target person with the MMSE score of 28 or higher is a “cognitively healthy,” the target person with MMSE score of 24 to 27 is a “mild cognitive impairment suspected,” and the target person with MMSE score of 23 or lower is a “dementia suspected”.

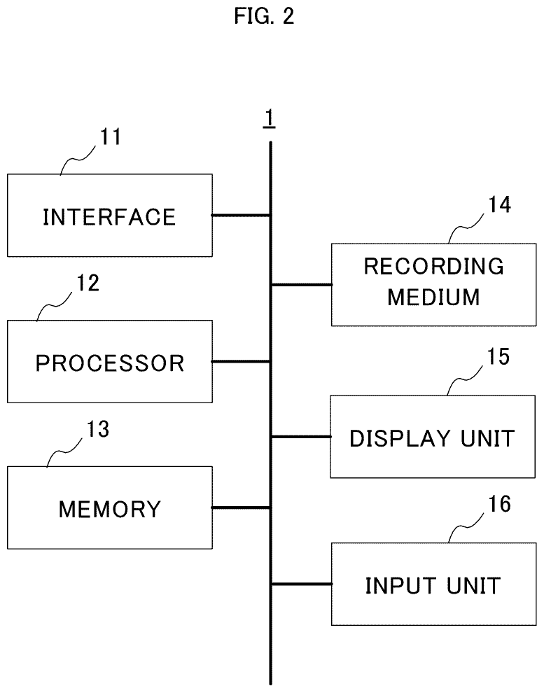

FIG. 2 is a block diagram illustrating an example of a hardware configuration of the cognitive function estimation device 1. As illustrated, the cognitive function estimation device 1 includes an interface (Interface) 11, a processor 12, a memory 13, a recording medium 14, a display unit 15, and an input unit 16.

The interface 11 exchanges data with the camera 2. The interface 11 is used to receive the video D1 from the camera 2. Also, the interface 11 is used when the cognitive function estimation device 1 transmits and receives data to and from a predetermined device connected by wire or wireless connections.

The processor 12 is a computer such as a CPU (Central Processing Unit) and controls the entire cognitive function estimation device 1 by executing a program prepared in advance. Incidentally, as the processor 12, a CPU, a GPU (Graphics Processing Unit), a DSP (Digital Signal Processor), a MPU (Micro Processing Unit), a FPU (Floating Point number Processing Unit), a PPU (Physics Processing Unit), a TPU (Tensor Processing Unit), quantum processor, microcontroller, or a combination thereof can be used.

The memory 13 consist of a ROM (Read Only Memory) and a RAM (Random Access Memory). The memory 13 stores programs executed by the processor 12. The memory 13 is also used as a working memory during various processes performed by the processor 12.

The recording medium 14 is a non-volatile and non-transitory recording medium such as a disk-shaped recording medium or a semiconductor memory and is configured to be detachable from the cognitive function estimation device 1. The recording medium 14 records various programs executed by the processor 12. In a case where the cognitive function estimation device 1 executes the cognitive function estimation process, the program recorded in the recording medium 14 is loaded into the memory 13 and executed by the processor 12.

The display unit 15, for instance, an LCD (Liquid Crystal Display), and displays a predetermined image. The input unit 16 is a keyboard, a mouse, a touch panel, or the like, and is used by an operator who manages the cognitive function estimation device 1.

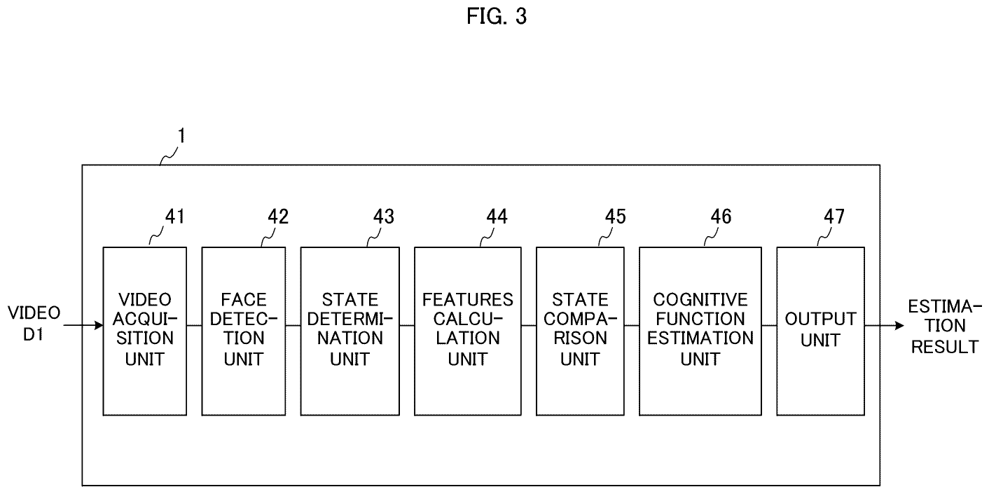

FIG. 3 is a block diagram illustrating an example of the functional configuration of the cognitive function estimation device 1. The cognitive function estimation device 1 functionally includes a video acquisition unit 41, a face detection unit 42, a state determination unit 43, a features calculation unit 44, a state comparison unit 45, a cognitive function estimation unit 46, and an output unit 47. Note that the video acquisition unit 41, the face detection unit 42, the state determination unit 43, the features calculation unit 44, the state comparing unit 45, the cognitive function estimation unit 46, and the output unit 47 are realized by corresponding programs which are executed by the processor 12.

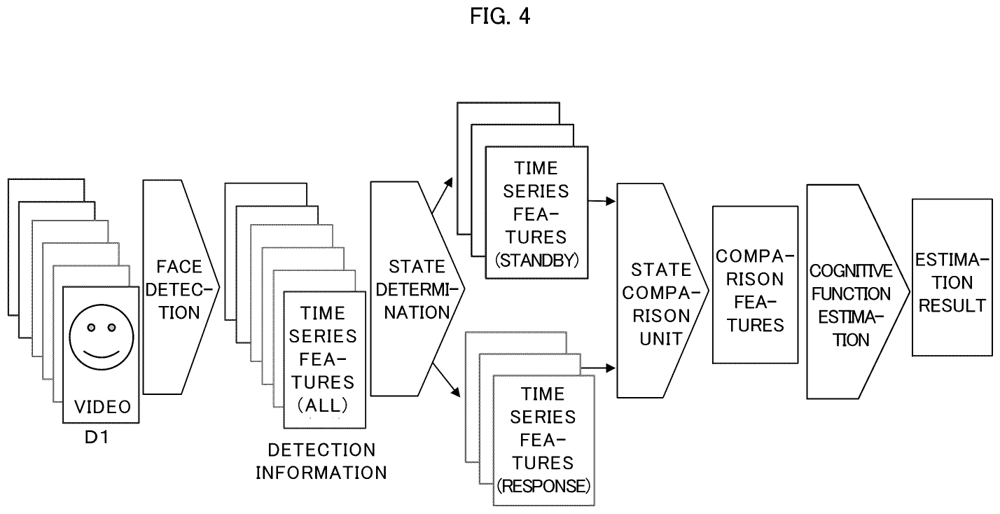

FIG. 4 is a diagram schematically illustrating a cognitive function estimation process performed by the cognitive function estimation device 1. As shown in FIG. 4, the cognitive function estimation device 1 first acquires detection information that is a time series features by detecting a position of the face of the target person and feature points of components of the face from the video D1 that is the time series information. The cognitive function estimation device 1 determines the state of the target person based on the detection information and classifies each predetermined section forming the video D1 for each state. In the present disclosure, each predetermined section forming the video D1 is also referred to as a “scene.” The cognitive function estimation device 1 calculates time series features of each state from the scene classified, and calculates comparison features by comparing the features among respective states. Then, the cognitive function estimation device 1 estimates the cognitive function of the target person based on the comparison features and outputs the estimation result.

The video acquisition unit 41 acquires, from the camera 2, the video D1 capturing the face of the target person in interaction with the healthcare worker. For the camera 2, any camera can be applied such as a surveillance camera, a smartphone, or the like, in a case where the camera is capable of capturing the interaction between the healthcare worker and the target person. Also, the interaction between the medical worker and the target person is not limited to the interaction facing directly, and the interaction may be an interaction through the network such as an on-line medical process.

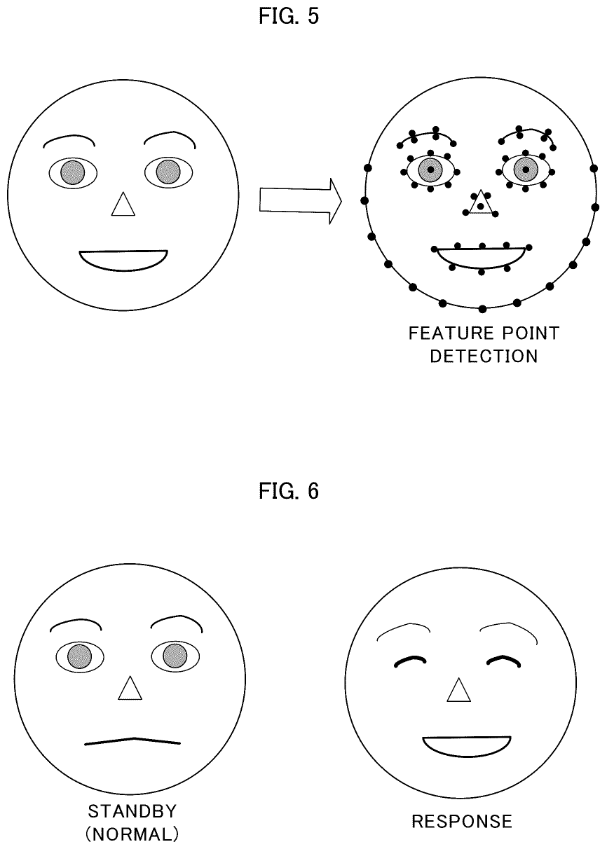

The face detection unit 42 acquires detection information that are time series features by detecting the position of the face of the target person and the feature points of the components of the face from the video D1. FIG. 5 is a diagram illustrating the feature points of the components of the face which are represented by black dots. As shown in FIG. 5, the face detection unit 42 detects, from the video D1, the position of the face of the target person and the feature points of eyebrows, eyes, a nose, a mouth, and outlines forming the face, and acquires time series information of coordinates indicating the position and the feature points of the face as the detection information.

The state determination unit 43 determines the state of the target person in accordance with the features defined from the face portion based on the detection information, and classifies the scene for each state. The state may be, for instance, a standby state in which the target person is waiting or a response state in which the target person conducts a predetermined task. The standby state may be, for instance, a state in which the target person is waiting while hearing to the healthcare worker and a normal state. On the other hand, the response state may be a state in which the target person responds to a stimulus such as a diagnosis or a question from the healthcare worker, such as a task of some kind, or a state other than normal. FIG. 6 illustrates examples of facial expressions of the target person in the standby state and the response state. Since the standby state and the response state always occur during the interaction, the video D1 used for interactions basically consists of two scenes: a scene of the standby state and a scene of the response state.

Specifically, the state determination unit 43 detects and aligns the face images of the target person from the video D1 based on the feature points of the face included in the detection information, and extracts features related to the muscles around the mouth, which exhibit the greatest variability among all facial expressions, using the feature points of the face on the aligned coordinates. The features related to the muscles around the mouth includes, for instance, a mouth corner distance indicating each distance from a mouth center to both ends of the mouth corners, and a mouth corner variation speed calculated from a variation amount of the mouth corner distance. Then, the state determination unit 43 determines the state of the target person according to the features extracted. For instance, in a case where the mouth corner distance is extracted as the features, the state determination unit 43 determines that the mouth represents the standby state if the mouth is closed for a certain period of time, and determines that the mouth is in the response state if the mouth is not closed for a certain period of time. In addition, in a case where the mouth corner variation speed is extracted as the features, the state determination unit 43 determines that the mouth corner variation speed is less than a threshold value as the standby state, and determines that the state is the response state if the mouth corner variation speed is equal to or more than the threshold value. The state determination unit 43 may perform a state determination using a machine learning model that is trained to estimate the state of the target person based on the features input.

In a case where the state of the target person is determined based on the detection information, the state determination unit 43 classifies the scene of the video D1 into the scene of the standby state or the scene of the response state. Scenes, which do not belong to any of the scenes in the standby state and scene in the response state, are not be used to estimate the cognitive function.

Although the state determination unit 43 uses the features originating from the muscles around the mouth in order to determine the state of the target person, it is not limited to this manner, but may be used by extracting the features related to the muscles around the eyes such as closing the eyes for a certain period of time, the features related to the whole face movements such as nodding, the features related to a facial expression such as a neutral expression and facial expressions other than the neutral expression. For instance, in a case of using the features related to the muscle around the eye, the state determination unit 43 determines that the target person is in the standby state if the eyes are closed for a certain period of time, or is in the response state if the eyes are not closed for a certain period of time. Moreover, in a case of using features related to the movement of the whole face, the state determination unit 43 determines the standby state if the movement frequency of the nodding is equal to or greater than the threshold value, and the response state if the movement frequency of the nodding is less than the threshold value. Furthermore, in a case of applying the features related to the facial expression of the whole face, the state determination unit 43 determines that the facial expression is in the standby state with respect to the neutral expression, and determines that the facial expression is in the response state with respect to the facial expressions other than neutral expression.

For each state, the features calculation unit 44 calculates the variation amount in the facial expression in time series in the scene classified for each state, and calculates the features associated with the variation amount in the facial expression in each state. Specifically, the features calculation unit 44 calculates the variation amount in the facial expression in the scene of the standby state, and calculates the features in the standby state. Also, the features calculation unit 44 calculates the variation amount in the facial expression in the scene of the response state, and calculates the features of the response state.

Here, the variation amount in the facial expression will be described. The variation amount in the facial expression may be, for instance, the variation amount calculated based on information related to the movements of the muscle around the mouth such as the mouth corner distance and the mouth corner variation speed, the variation amount calculated based on the information related to the movements of the muscle around the eyes such as an eye closure rate, the variation amount calculated based on the information related to the movements of the whole face such as the nodding, the variation amount calculated from the information related to the facial expression of the whole face, or the like. Thus, it is possible for the cognitive function estimation device 1 to apply a physical variation amount in the facial expression as features in order to estimate the cognitive function.

The state comparison unit 45 calculates comparison features by comparing the features of respective states. In detail, the state comparison unit 45 calculates the comparison features by taking the ratio of the features in the standby state to those in the response state.

The cognitive function estimation unit 46 estimates the cognitive function of the target person based on the comparison features. In other words, the cognitive function estimation unit 46 estimates a cognitive decline based on the difference between facial expression variations in the standby state and the response state. In the estimation of the cognitive function, compared to a case where predetermined features are calculated from all scenes in the video D1, if the predetermined features are calculated for each state by classifying the video D1 into the scene in the standby state or the scene in the response state, a correlation between the score of MMSE for estimating the cognitive function and the features can be increased.

In detail, the cognitive function estimation unit 46 estimates an evaluation of the cognitive function based on the comparison features in accordance with experimental result described below. For instance, the evaluation may be a classification based on a value corresponding to the score of the MMSE calculated based on the comparison features or a simple classification such as the “cognitively healthy” or the “suspected cognitive decline” may be classified, and thus, the evaluation can be arbitrarily set. In the present disclosure, the score of the MMSE is applied, but the evaluation is not limited thereto, and a score from any test that evaluates the cognitive function, such as MoCA-J (Japanese version of Montreal Cognitive Assessment), may be applied.

In one specific example, in a case where the features in the standby state indicate an average mouth corner distance in the scene in the standby state and the features in the response state indicate an average mouth corner distance in the scene in the response state, the comparison features indicate a ratio of the average mouth corner distance in the standby state to the average mouth corner distance in the response state. As a specific example, in a case of comparing a person who has not advanced the cognitive decline with a person who has advanced the cognitive decline, the person without an advanced cognitive decline tends to have a larger mouth corner distance in the response state than that in the standby state compared. This large variation of the mouth corner distance represents that the variation amount in the facial expression around the mouth is large and that the muscles around the mouth are not attenuated. Therefore, in a case where the mouth corner distance in the response state tends to be smaller according to the comparison features, the cognitive function estimation unit 46 estimates that cognitive decline is likely to be advanced. At this time, the cognitive function estimation unit 46 may calculate a value corresponding to a score of MMSE based on the comparison features. On the other hand, in a case where there is no particular problem in the comparison features, the cognitive function estimation unit 46 estimates that the cognitive function is healthy.

In another specific example, the comparison features are defined as a ratio of an eye closure rate in the standby state to an eye closure rate in the response state. The eye closure rate represents a percentage of a value in which a degree of an eye opening, which is a degree to which the eyes are open, is lower than a threshold value. As a specific example, a person with the advanced cognitive decline tends to have a greater eye closure rate in the response state than a person without the advanced cognitive decline. Therefore, in a case where the comparison features show a tendency for a larger eye closure rate in the response state, the cognitive function estimation unit 46 estimates that the cognitive function is likely to be being declined. On the other hand, in a case where there is no particular problem with the comparison features, the cognitive function estimation unit 46 estimates that the cognitive function is healthy.

In another specific example, the comparison features are defined as a ratio of an average mouth corner variation speed in the standby state to an average mouth corner variation speed in the response state. As a specific example, the person with the advanced cognitive decline tends to have a slower mouth corner variation speed in the response state than the person without the advanced cognitive decline. Therefore, in a case where the mouth corner variation speed in the response state tends to be slower according to the comparison features, the cognitive function estimation unit 46 estimates that the cognitive function is likely to be in the advanced decline. On the other hand, in a case where there is no particular problem with the comparison features, the cognitive function estimation unit 46 estimates that the cognitive function is healthy.

In another specific example, the comparison features are defined as a ratio of a frequency or intensity of the expression variation in the standby state to a frequency or intensity of the expression variation in the response state. As a specific example, the person with the advanced cognitive decline tends to show less frequency or less intensity of the facial expression variation than the person without the advanced cognitive decline. Therefore, the frequency or intensity of the facial expression variation tends to be less according to the comparison features, the cognitive function estimation unit 46 estimates that the cognitive function is likely to be declining more. On the other hand, in a case where there is no particular problem with the comparison features, the cognitive function estimation unit 46 estimates that the cognitive function is healthy.

Note that the cognitive function of the target person may be estimated using a cognitive function estimation model that is the machine learning model. For instance, if the comparison features are input, the cognitive function estimation unit 46 may build the cognitive function estimation model that has been optimized to output the evaluation of the cognitive function. To construct (generate) the cognitive function estimation model, labeled data are used. The labeled data are data in which input data to be input in learning of the cognitive function estimation model are associated with a correct output corresponding to the input data. The input data are various comparison features, and the correct output are evaluations of cognitive functions. The cognitive function estimation unit 46 trains the cognitive function estimation model so as to output the evaluation of the cognitive function based on the comparison features input as the input data. As a method of machine learning, for instance, a model using a neural network, and the like are exemplified. According to this method, the cognitive function estimation unit 46 can use the evaluation of the cognitive function which the cognitive function estimation model outputs, as an estimation result.

The output unit 47 outputs the estimation result by the cognitive function estimation unit 46. In detail, the output unit 47 provides the estimation result of the cognitive function estimation unit 46 to the healthcare worker by displaying or transmitting to a predetermined terminal.

Moreover, in the configuration described above, the video acquisition unit 41, the face detection unit 42, the state determination unit 43, the features calculation unit 44, the state comparison unit 45, and the cognitive function estimation unit 46 of the cognitive function estimation device 1 correspond to examples of the video acquisition means, a body part detection means, the state determination means, the features calculation means, the state comparison means, and the cognitive function estimation means of the present disclosure, respectively.

### Cognitive Function Estimation Process

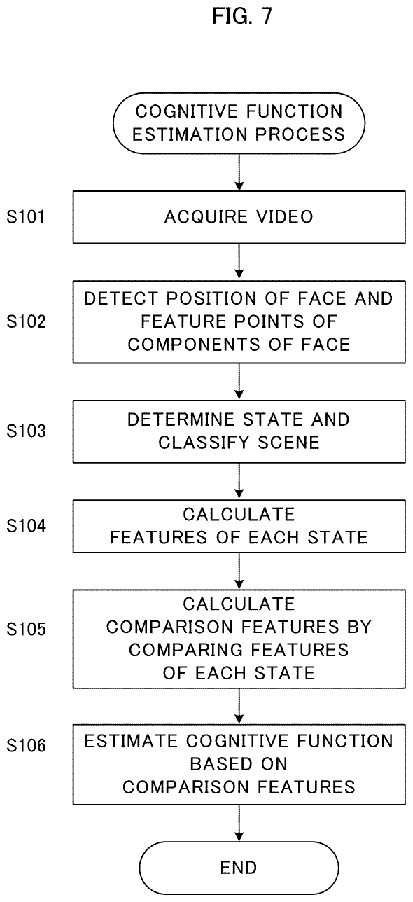

Next, a cognitive function estimation process by the cognitive function estimation device 1 will be described. FIG. 7 is a flowchart of the cognitive function estimation process performed by the cognitive function estimation device 1. This cognitive function estimation process is realized by executing a corresponding program prepared in advance by the processor 12 shown in FIG. 2.

First, the cognitive function estimation device 1 acquires the video D1 obtained by capturing facial images of the target person (step S101). Next, the cognitive function estimation device 1 acquires detection information that are time series features by detecting the position of the face of the target person and the feature points of the components of the face from the video D1 (step S102). Next, based on the detection information, the cognitive function estimation device 1 determines the state of the target person and classifies the scene for each state (step S103). Next, the cognitive function estimation device 1 calculates the features of the state from each scene classified (step S104). Subsequently, the cognitive function estimation device 1 calculates comparison features comparing the features among the respective states (step S105), and estimates the cognitive function of the target person based on the comparison features (step S106). Thus, the cognitive function estimation device 1 outputs the estimation result of the cognitive function and terminates the cognitive function estimation process.

In the present example embodiment, the cognitive function is estimated using the features based on the variation amount in the facial expression, but is not limited thereto, and the features based on voice may be used. In detail, the comparison features are defined as a ratio of an average response speed by the voice in the standby state to an average response speed by the voice in the response state. Experiments showed that persons with the advanced cognitive decline had a slower response speed than persons without the advanced cognitive decline. Therefore, in a case where the response speed of the response state tends to be slow according to the comparison features, the cognitive function estimation unit 46 estimates that the cognitive function is likely to be in advanced decline.

Moreover, the features for determining the state of the target person may be applied to the features to be calculated for the scene of each state, or the features to be calculated for the scene of each state may be applied to the features for determining the state of the target person, and thus the features to be applied can be arbitrarily set.

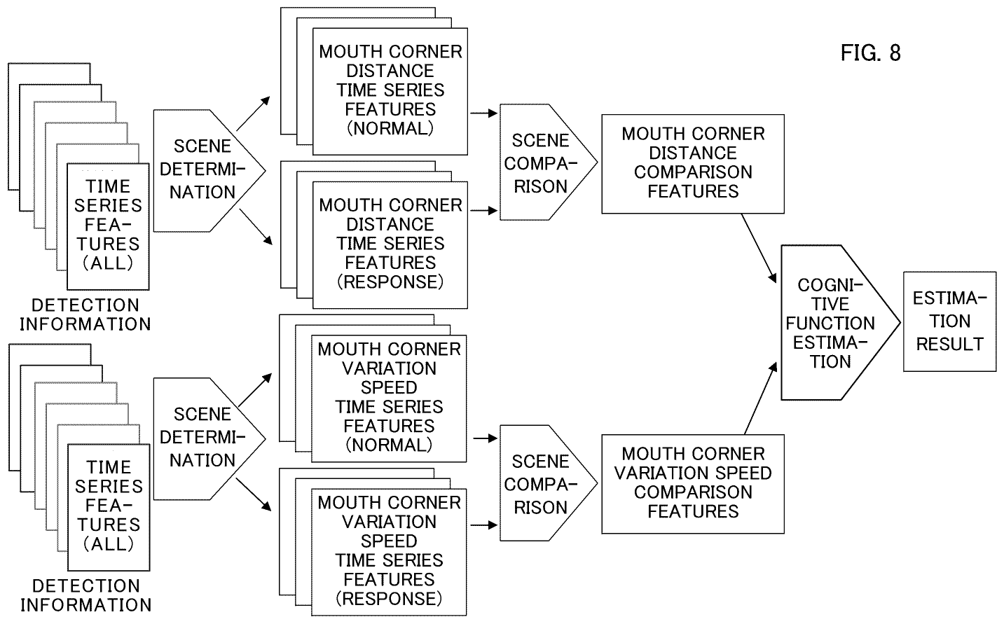

FIG. 8 is a diagram schematically illustrating the recognition function estimation process using comparison features for a plurality of types for each state. As shown in FIG. 8, the cognitive function estimation device 1 determines the state of the target person according to the predetermined features based on the detection information from one video D1 corresponding to one target person, and classifies the scene for each state. At this time, the features to be applied to the determination of the state may be the same or may be different. The cognitive function estimation device 1 calculates the features of each state from the classified scene, respectively. At this time, the cognitive function estimation device 1 calculates a plurality of combinations of the features for each state using a plurality of features. For instance, as shown in FIG. 8, the cognitive function estimation device 1 calculates, as the features, the average mouth corner distance in the standby state and the average mouth corner distance in the response state, and also calculates, as the features, the average mouth corner variation speed in the standby state and the average mouth corner variation speed in the response state.

The cognitive function estimation device 1 calculates the comparison features by comparing the average mouth corner distance in the standby state with the average mouth corner distance in the response state, calculates the comparison features by comparing the average mouth corner variation speed in the standby state with the average mouth corner variation speed in the response state, and estimates the cognitive function based on two sets of the comparison features. Thus, the cognitive function estimation device 1 can improve accuracy of the estimation result by estimating the cognitive function based on a plurality of sets of the comparison features.

A technology for detecting the dementia using the facial expressions of the target person extracted from a video is effective as a trend and a point of focus, but has a problem that facial expression manifestations vary widely from person to person and are difficult to use as a diagnostic indicator. In addition, a technology for detecting dementia uses an output of a machine learning model to which a video prepared is input, as the basis for the decision, and since a process for outputting a result from the video which is input is unclear, it is difficult for the healthcare worker to interpret a basis for the diagnosis of the dementia. In order for the healthcare worker to use the detection of the dementia using the video as the basis for a dementia diagnosis, it is necessary to clarify which scene from the video has been used to detect the disease.

According to the cognitive function estimation system 100 of the present disclosure, by classifying and using images of the video D1 into any of a plurality of scenes belonging to the standby state or the response state, it is possible to clarify which scene is used to estimate the cognitive function, and at the same time, it is possible to detect the cognitive decline at an early stage while reducing the psychological and economic burdens of the target person. Moreover, according to the cognitive function estimation system 100, not only short-time diagnostic, task, and test scene videos cropped for research purposes, but also a long-time video D1, which contain scenes similar to natural interaction, can be used to extract scenes for diagnosis and estimate the cognitive function.

Moreover, according to the cognitive function estimation system 100 of the present disclosure, the comparison features calculated by the cognitive function estimator 1 are the basis used in estimating the cognitive function. Therefore, in addition to face-to-face examinations with the target person, it is possible for the healthcare worker to quantitatively capture the mouth corner distance, mouth corner variation speed, and the eye closure rate, which provide the basis for diagnosing the cognitive function.

As described above, according to the cognitive function estimation device 1 of the present disclosure, one video D1 can be divided into various states or scenes to be used for estimating the cognitive function. Moreover, even with a single video D1, the feature values for the standby state, response state, and comparison can be varied in various ways depending on the defined feature values. Furthermore, it is possible to increase the accuracy of the estimation result because the cognitive function estimation device 1 uses a feature quantity associated with a physical quantity, i.e. the variation amount in the facial expression, which is more granular than the emotions conventionally used, to estimate the cognitive function.

Moreover, in a case where the video D1 is the natural interaction, if features are calculated based on emotion alone, as in conventional technology, the estimation result may change depending on a topic or mood of the day, and the emotion to be analyzed may not occur during the interaction in the first place. This problem of individual differences can be solved by calculating the features using the physical quantity representing the variation amount in facial muscles as an indicator, based on the detection information including the position of the face and the feature points of the components of the face, as in the cognitive function estimation device 1 of this disclosure.

### First Modification

In the above example embodiment, the portion of the body used for estimating the cognitive function is a face, and the detection information concerning the face of the target person is acquired from the video D1, but the present disclosure is not limited thereto, and the portion of the body can be arbitrarily set.

In one specific example, the features are calculated based on a movement of a finger or a height of a hand, and the features calculated can be applied to the state determination of the target person or can be used as the features to be calculated for the scene of each state. In this instance, the cognitive function estimation device 1 acquires the detection information including the position of the hand or the finger and the features of the hand or the finger from the video D1.

In another specific example, the features are calculated based on a movement of each foot such as subtle trembling, and the calculated features can be applied to the state determination of the target person or can be used as the features to be calculated for the scene of each state. In this instance, the cognitive function estimation device 1 acquires the detection information including the position of each foot and the feature points of each foot from the video D1.

In another specific example, the features are calculated based on a posture such as a forward-leaning posture, and the calculated features can be applied to the state determination of the target person, or can be features to be calculated for the scene of each state. In this case, the cognitive function estimation device 1 acquires the detection information including the position of a waist and a back and the feature points of the waist and the back from the video D1.

### Second Modification

In the example embodiment described above, the cognitive function estimation device 1 determines the standby state and the response state of the target person based on the detection information concerning the face, and classifies a sequential scene or a non-sequential scene for each state from the video D1. However, the states are not limited to these two states, may include three or more states such as the standby state, a first response state, a second response state, etc. In this case, for instance, the standby state is defined as a neutral expression, the first response state is defined as a smiling expression, and the second response state is defined as a surprising expression, and the features related to the facial expression of the entire face is used to discriminate between three states, and scenes of respective states are classified from the video D1. The cognitive function estimation device 1 calculates the features of each state and calculates a plurality of comparison features by comparing the features between all states which are considered valid. Thus, the number of states used to estimate the cognitive function can be arbitrarily set.

### Third Modification

In the above example embodiment, the cognitive function estimation device 1 estimates the cognitive function of the target person based on the comparison features; however, the present disclosure is not limited thereto and may be configured to perform only the processing up to the calculation of the comparison features and support the estimation of the cognitive function. In this case, the cognitive function estimation device 1 provides the comparison features to the healthcare worker, and allows healthcare workers to diagnose the cognitive function on the basis of the comparison features. In other words, the comparison features may be used as information to support a diagnostic rationale of the healthcare worker.

In addition, some or all of the above embodiments (including modifications,

same hereinafter) may also be described as the following supplementary notes, but not limited thereto.

### Supplementary Note 1

A cognitive function estimation device comprising:
a video acquisition means configured to acquire a video of a target person;
a body part detection means configured to detect a specific body part forming a body of the target person from the video, and acquire detection information concerning the body part;
a state determination means configured to classify each section of the video for each state by determining the state of the target person based on the detection information;
a features calculation means configured to calculate, for each state, a variation amount of a specific body part in each section of the video which is classified, and calculate features associated to the variation amount for each state; and
a state comparison means configured to calculate comparison features, which are features related to comparison between states, by comparing features of respective states.

### Supplementary Note 2

The cognitive function estimation device according to supplementary note 1, further comprising a recognition cognitive function estimation means configured to estimate a cognitive function of the target person based on the comparison features.

### Supplementary Note 3

The cognitive function estimation device according to supplementary note 2, wherein
the specific body part is a face,
the body part detection means acquires detection information by detecting a position of the face of the target person and feature points of components of the face from the video, and
the features calculation means calculates, for each state, a variation amount in a facial expression in each section in the video which is classified, and calculates features associated with the variation amount in the facial expression for each state.

### Supplementary Note 4

The cognitive function estimation device according to supplementary note 3, wherein
the state determination means determines whether the target person is in a first state or a second state based on the detection information, and classifies the video into a section of the first state and a section of the second state,
the features calculation means calculates the variation amount in the facial expression in each of sections of the first state and the second state, and calculates, for each section, features associated with the variation amount calculated, and
the state comparison means calculates comparison features being features resulted from comparing features of the section of the first state and features of the section of the second state.

### Supplementary Note 5

The cognitive function estimation device according to supplementary note 4, wherein the first state is a standby state in which the target person is waiting and the second state is a response state in which the target person conducts a predetermined task.

### Supplementary Note 6

The cognitive function estimation device according to supplementary note 5, wherein
the features calculation means calculates features for each of a plurality of types for each state,
the state comparison means calculates the comparison features for each of the plurality of types by comparing features of each of the plurality of types of the standby state and features of each of the plurality of types of the response state, and
the cognitive function estimation means estimates the cognitive function of the target person based on the comparison features of each of the plurality of types.

### Supplementary Note 7

The cognitive function estimation device according to supplementary note 4, wherein the variation amount in the facial expression indicates a variation amount calculated based on information related to a movement of muscles around a mouth.

### Supplementary Note 8

The cognitive function estimation device according to supplementary note 2, wherein the cognitive function estimation means estimates the cognitive function of the target person, by using a machine learning model trained and optimized to output an evaluation of the cognitive function in response to an input of the comparison features.

### Supplementary Note 9

A cognitive function estimation method performed by a cognitive function estimation device, comprising:
acquiring a video of a target person;
detecting a specific body part forming a body of the target person from the video, and acquire detection information concerning the body part;
classifying each section of the video for each state by determining the state of the target person based on the detection information;
calculating, for each state, a variation amount of a specific body part in each section of the video which is classified, and calculating features associated to the variation amount for each state; and
calculating comparison features, which are features related to comparison between states, by comparing features of respective states.

### Supplementary Note 10

A program causing a computer to execute processing of:
acquiring a video of a target person;
detecting a specific body part forming a body of the target person from the video, and acquire detection information concerning the body part;
classifying each section of the video for each state by determining the state of the target person based on the detection information;
calculating, for each state, a variation amount of a specific body part in each section of the video which is classified, and calculating features associated to the variation amount for each state; and
calculating comparison features, which are features related to comparison between states, by comparing features of respective states.

While the present disclosure has been described with reference to the example embodiments and examples, the present disclosure is not instance limited to the above example embodiments and examples. Various changes which can be understood by those skilled in the art within the scope of the present disclosure can be made in the configuration and details of the present disclosure.

This application is based upon and claims the benefit of priority from Japanese Patent Application 2024-049287, filed on Mar. 26, 2024, the disclosure of which is incorporated herein in its entirety by reference.

### DESCRIPTION OF SYMBOLS

1 Cognitive function estimation device
2 Camera
11 Interface
12 Processor
13 Memory
14 Recording medium
15 Display unit
16 Input unit
41 Video acquisition unit
42 Face detection unit
43 State determination unit
44 Features calculation unit
45 State comparison unit
46 Cognitive function estimation unit
47 Output unit
100 Cognitive function estimation system

1. A cognitive function estimation device comprising:
at least one memory configured to store instructions; and
at least one processor configured to execute the instructions to:
acquire a video of a target person;
detect a specific body part forming a body of the target person from the video, and acquire detection information concerning the body part;
classify each section of the video for each state by determining the state of the target person based on the detection information;
calculate, for each state, a variation amount of a specific body part in each section of the video which is classified, and calculate features associated to the variation amount for each state; and
calculate comparison features, which are features related to comparison between states, by comparing features of respective states.

2. The cognitive function estimation device according to claim 1, the processor is further configured to estimate a cognitive function of the target person based on the comparison features.

3. The cognitive function estimation device according to claim 2, wherein
the specific body part is a face,
the processor acquires detection information by detecting a position of the face of the target person and feature points of components of the face from the video, and
the processor calculates, for each state, a variation amount in a facial expression in each section in the video which is classified, and calculates features associated with the variation amount in the facial expression for each state.

4. The cognitive function estimation device according to claim 3, wherein
the processor determines whether the target person is in a first state or a second state based on the detection information, and classifies the video into a section of the first state and a section of the second state,
the processor calculates the variation amount in the facial expression in each of sections of the first state and the second state, and calculates, for each section, features associated with the variation amount calculated, and
the processor calculates comparison features being features resulted from comparing features of the section of the first state and features of the section of the second state.

5. The cognitive function estimation device according to claim 4, wherein the first state is a standby state in which the target person is waiting and the second state is a response state in which the target person conducts a predetermined task.

6. The cognitive function estimation device according to claim 5, wherein
the processor calculates features for each of a plurality of types for each state,
the processor calculates the comparison features for each of the plurality of types by comparing features of each of the plurality of types of the standby state and features of each of the plurality of types of the response state, and
the processor estimates the cognitive function of the target person based on the comparison features of each of the plurality of types.

7. The cognitive function estimation device according to claim 4, wherein the variation amount in the facial expression indicates a variation amount calculated based on information related to a movement of muscles around a mouth.

8. The cognitive function estimation device according to claim 2, wherein the processor estimates the cognitive function of the target person, by using a machine learning model trained and optimized to output an evaluation of the cognitive function in response to an input of the comparison features.

9. A cognitive function estimation method performed by a cognitive function estimation device, comprising:
acquiring a video of a target person;
detecting a specific body part forming a body of the target person from the video, and acquire detection information concerning the body part;
classifying each section of the video for each state by determining the state of the target person based on the detection information;
calculating, for each state, a variation amount of a specific body part in each section of the video which is classified, and calculating features associated to the variation amount for each state; and
calculating comparison features, which are features related to comparison between states, by comparing features of respective states.

10. A program causing a computer to execute processing of:
acquiring a video of a target person;
detecting a specific body part forming a body of the target person from the video, and acquire detection information concerning the body part;
classifying each section of the video for each state by determining the state of the target person based on the detection information;
calculating, for each state, a variation amount of a specific body part in each section of the video which is classified, and calculating features associated to the variation amount for each state; and
calculating comparison features, which are features related to comparison between states, by comparing features of respective states.
